SEQUENCE LISTING (40 sequences)

SEQ ID NO: 1 - DNA, 1845 bp, Toxoplasma gondii
  CDS (1)..(1845)
atgcaactcccttcaagaaaggagtccggaacccccaacaatgcggcgtcaccaggcagcctaaagtcagcgagaaccacacgcgcttcgagtccctcgatggccgagtgttcgcctcgagtgcttgtcaactgcggtgtcgctccgactgagcaaggggcccatggtgagcataaaagcttggaagtctccactgacaactcagcttgcaaggaaccccggcgcatgctctggggttcaattctcatcctgagtttactgcttctcgggttctgggaccagctgtttccgccttcccagatgaaagatgtcgtgcgctctgtccacgaaggtgccgggaaacctggcgatgttgctggttctgcagagcttcctgcagaaaggtgtgtcgagggagagtggaagagggacaaggggggagagtctgcaccaggtgacgaccccgcgtgccaaaaaggcgccacacaagcgacgcctcgtccatctgaggaacacgcgggagcggcgaaaccaacccctgcaactccttccgtgttttcttcactccttggtgattggtcatctcccgtttccagctctccaaagagcgcgaatgtcaggcgtgtcgtgctagcgcttctcattcttgtctgcatgtactgtttcatccaggcgagagactgctcactcgttctcccgcaccccggcttctggcgtgtggtgcacggcatgtgcgtcgttcacttgattgtcatggtggtgctgctggtcgttgacgaagagacgggaagacacgcgttggagttgctgttccccgaaatcgcaggaaaaagagaggaaatttttgcggggacactagtggtagactgccgaatcaatgcaagcacgatccaccgacagttgacaagtgtgtggttcgtctcccacgttgttggctggcttgtaaagatggtcattctccgacactgggggttttgtctcatctactccctttgctttgaactaggcgaactgtcgttccactggctcgtccccgaactctgcgaatgctggtgggacagcatctttatcgatgcgctgctcagcaacgtttgcggcatgtttctgggggtcctcttcatgaagcttgtcaacctgtgccagtacgactggctaggccgctacccgcaggatcagaacgtttccattagtctgactccgtttgcatctgaagcttccgactggagcttctataaaacgccccgccatctgttcatgtctatcgtgctccttatgatctgtctcttttcggagctgaacgtcttctttcttatggcagcccttgacatccctgctgcgcactacagcaaccctctccgtaccttgtatatcgctctcctaggagttgccgcagctgcagagcactatgaattcaccacgaaccgccgcgacagaattggccggaataagtggttactcattatcattctctttgtcgaatggctggtgtgcgcaaaatacggagcgcgccggtacgctgcagggctccctccccccgacatttttgtgccgtggattgttagctccatcctcttttctctgtggtgttactgttactataccacagctgacctccgcacagacagtgggaaagggaagctgctggcgaagtctcccggcgggaaaggagcgggtgacgagagcgcaaatctggcagcagagtcgaaaatgacaaagcggtcagctaagcagtgtccagcggcttcctggcaggatcggatcaagctgctcgttctaacattaccccctcaggcatgctttatgccgctgctgtatctctcgaaattctactttttcgactacgtgaaaatcgaacgaagtcagtga

SEQ ID NO: 2 - protein, 614 aa, Toxoplasma gondii
MQLPSRKESGTPNNAASPGSLKSARTTRASSPSMAECSPRVLVNCGVAPTEQGAHGEHKSLEVSTDNSACKEPRRMLWGSILILSLLLLGFWDQLFPPSQMKDVVRSVHEGAGKPGDVAGSAELPAERCVEGEWKRDKGGESAPGDDPACQKGATQATPRPSEEHAGAAKPTPATPSVFSSLLGDWSSPVSSSPKSANVRRVVLALLILVCMYCFIQARDCSLVLPHPGFWRVVHGMCVVHLIVMVVLLVVDEETGRHALELLFPEIAGKREEIFAGTLVVDCRINASTIHRQLTSVWFVSHVVGWLVKMVILRHWGFCLIYSLCFELGELSFHWLVPELCECWWDSIFIDALLSNVCGMFLGVLFMKLVNLCQYDWLGRYPQDQNVSISLTPFASEASDWSFYKTPRHLFMSIVLLMICLFSELNVFFLMAALDIPAAHYSNPLRTLYIALLGVAAAAEHYEFTTNRRDRIGRNKWLLIIILFVEWLVCAKYGARRYAAGLPPPDIFVPWIVSSILFSLWCYCYYTTADLRTDSGKGKLLAKSPGGKGAGDESANLAAESKMTKRSAKQCPAASWQDRIKLLVLTLPPQACFMPLLYLSKFYFFDYVKIERSQ

SEQ ID NO: 3 - DNA, 1884 bp, Neospora caninum
  CDS (1)..(1884)
atgcaactccgcgagagaaagctcgcgggagcttttgacaaccaaggctcgccgcaccatctgaagccgcctgtgacgcctggctcttcatgctcctcagtcaacgagtgttcgcggcgggcgtcccatgactccaacgcggcctcggtttaccgcgaacatgcagcccactatgcgcagaagtttcgagccgccatcgactacacgactcgggaggaacctcgccagattctgtggggttttattcttgtcctgagcttgcttcttctcgggttccgtgaccagctgctgccgtcttcttctcaggtgaacctggtgcacagaggcgccggagaagctgttcagcccacggtctcgccaagtgagactgctgtcgctcctggcgcaaaacttcctggaaatgatgccacggtgaagtgcaccgcgggttcgcgaaagaagaacgacaacgaagatgttggagcagatgggggtgacgatccgaatgaaagctgtcagcgggctacaccagaggctcctcgtgtatctgagcagcagtcttctacgtccggtcgtactcaggttgctccttcggtgttttcttcaatctttggtggttcgtcctcggcctctgtctcttctcctctgagcacgaatgtcaggcgagtggtgactgggcttcttgtcctcgtctgcctgtactgtttcctccaggcgaaagatggtttgctcgttcgtccacaccctggtttctggcgcctggtacatggcgtttgcctggttcacttgattgttatggttgtgcttctggtcctcgacgtggagaccggactacttgttttggagttgttgttcccagaaattgcagggaaaaggaaagaaatttttgcaggaacacttgtgcttgattgccgaatcaacggagacacgatcaagcgtcagttaacgagtgtgtggttcatctcgcatgttgttggttggctagggaagatggtcattctcagaaattggggtctttgtcttctttactccatcttctttgaattaggagaattgtccttccactggcttgtcccagaactttgcgaatgctggtgggacagtatcttcatagatgctcttctgtccaatgtaagcggcatgcttctgggggccgtcttcatgaagtttatcaacatgcaccagtatgactggctaggccgccatccgctgtaccagaaggtcttcatgagtttgactcccttttcttccgagggctatgactggagcttctacaagaccccacggcacctgttcctgtccatcgcactgctcacgttttgcgttttcttggagttgaatgtcttcttccttatggccgcgcttgatatccctgcaacccactatatcaaccctctccgaacgctgtacctcactctactaggcgctgcggccgcccccgagcactacgaatataccatgttcagtcgagagagagttggccacaacgaatggttacttgttgtgatcctcagtgtcgaattgttggtgtgtgtcaagtatggggcaggccggtacaacgccgagacccctcccctcgatctagttataccgtgggttgctgcgctcagcctctttgctgcatggtgttactgttacttttcgacggctgacctccgcgggaacaatggcgaggaagggatcaaggcgcctgtgtcccacgcacgaagagggggagaagaaatggaaaatctggcagaagaaccgaaaaagacaaaacgatcagagaagcatcgagtgcattcctggcaagaccaggccaagctgcttgtcattacagtgcctcctcaggcgtgttttatccctctgctgtatcttatgaaattctacttttacgactacgtcaaaatcgagcgcaatcagtga

SEQ ID NO: 4 - protein, 627 aa, Neospora caninum
MQLRERKLAGAFDNQGSPHHLKPPVTPGSSCSSVNECSRRASHDSNAASVYREHAAHYAQKFRAAIDYTTREEPRQILWGFILVLSLLLLGFRDQLLPSSSQVNLVHRGAGEAVQPTVSPSETAVAPGAKLPGNDATVKCTAGSRKKNDNEDVGADGGDDPNESCQRATPEAPRVSEQQSSTSGRTQVAPSVFSSIFGGSSSASVSSPLSTNVRRVVTGLLVLVCLYCFLQAKDGLLVRPHPGFWRLVHGVCLVHLIVMVVLLVLDVETGLLVLELLFPEIAGKRKEIFAGTLVLDCRINGDTIKRQLTSVWFISHVVGWLGKMVILRNWGLCLLYSIFFELGELSFHWLVPELCECWWDSIFIDALLSNVSGMLLGAVFMKFINMHQYDWLGRHPLYQKVFMSLTPFSSEGYDWSFYKTPRHLFLSIALLTFCVFLELNVFFLMAALDIPATHYINPLRTLYLTLLGAAAAPEHYEYTMFSRERVGHNEWLLVVILSVELLVCVKYGAGRYNAETPPLDLVIPWVAALSLFAAWCYCYFSTADLRGNNGEEGIKAPVSHARRGGEEMENLAEEPKKTKRSEKHRVHSWQDQAKLLVITVPPQACFIPLLYLMKFYFYDYVKIERNQ

SEQ ID NO: 5 - DNA, 1476 bp, Eimeria tenella
  CDS (1)..(1476)
atgcgggtgagccgcgggcaggcgccgacgggggggcccggcggggcccccgcggcgctgtcgccgtgggggcgtctgtgggccgctgcctggcgccccgcggcggcggccgccgctgctgctgcggagggggcggccgctgcggccgcgtcgggcgggggggcctcgggggccccccgcgggcgggcagggggccccccggggtgggggctggggagcgtcggggcgggggcggggctgctgctgctgctggcgggcgcgctgccgccgctgctggcgagctgggggccggcggcgcaggggctgtcggtggcggcgctgccggcggcgcagcagcaggagctgcgggcgctgctgctgcggagcctgggctcgacggcgctgccgctgtgcctctacctcgccttcctgctgcagcaggcgcaggtgcagcagccgcaggcgcagcaggcgctggcgtggagcgcggtgcacggcgcgggcgcgtgctacctgctgctgctgctgctgctgctggcgctgcccgcgcggctctgccgcgaactgctgcagctgctgctgcccggctgctgcagcggcggcaacgtcttcaccggcacgctcatcctcgactgctcgctgcgccgcagcacgctgctgcgccagctgcgctcgccctggttcgccagccacgccctcggctggttcctcaagatgagcctctaccgcagctggccctgcgccctcgcgctctccctgcttttcgaggccgcagaggcttcgctgcactggctgctgcccgagttccaggagtgctggtgggacagcgtggtgctggacgcggttctgtcgaacctgctggggatgactccgcggcacttggccatgacgggactgctcctcgggctctccttgctcgcggagctcaatgttttcttcttgatgacggccctggacctgcacgccacgcactgggtcaacccgctccggctgctgctcctcgggctgctggccttccccgcggtcgcggagttctacgcccagctccagcgcagcagcagcagcagcagcagcagcagcggcggcggcgtggggcccaacgtctttctttttgtcttcattttggcggcagaaaccctcgttgctgccaagtacggcagagacagattccactacaagagcccccccaaagatgtcctgcttccctggatcctcgctgctgctctcttcgccgcctggtgctgctcctacttcagccagcagcaggggggcccccctcccccggggggcccccaggcgcaggggggccccccgggggaggacgcggagaaggcttgcgagggggcgtgtggggtgtttaggactttgaacaacttcaaacggtctgctgccagactgctcctcaaaatgccgccccaagcctgcttcattcctcttctttacttggccaaaaactactactatggagaaccccacaccccacagccagctccctag

SEQ ID NO: 6 - protein, 491 aa, Eimeria tenella
MRVSRGQAPTGGPGGAPAALSPWGRLWAAAWRPAAAAAAAAAEGAAAAAASGGGASGAPRGRAGGPPGWGLGSVGAGAGLLLLLAGALPPLLASWGPAAQGLSVAALPAAQQQELRALLLRSLGSTALPLCLYLAFLLQQAQVQQPQAQQALAWSAVHGAGACYLLLLLLLLALPARLCRELLQLLLPGCCSGGNVFTGTLILDCSLRRSTLLRQLRSPWFASHALGWFLKMSLYRSWPCALALSLLFEAAEASLHWLLPEFQECWWDSVVLDAVLSNLLGMTPRHLAMTGLLLGLSLLAELNVFFLMTALDLHATHWVNPLRLLLLGLLAFPAVAEFYAQLQRSSSSSSSSSGGGVGPNVFLFVFILAAETLVAAKYGRDRFHYKSPPKDVLLPWILAAALFAAWCCSYFSQQQGGPPPPGGPQAQGGPPGEDAEKACEGACGVFRTLNNFKRSAARLLLKMPPQACFIPLLYLAKNYYYGEPHTPQPAP

SEQ ID NO: 7 - DNA, 18 bp, Artificial Sequence
  Synthetic primer
atgcaactcccttcaaga

SEQ ID NO: 8 - DNA, 25 bp, Artificial Sequence
  Synthetic primer
tcactgacttcgttccattttcacg

SEQ ID NO: 9 - DNA, 20 bp, Artificial Sequence
  Synthetic primer
atgtgtcggggaccgccgct

SEQ ID NO: 10 - DNA, 21 bp, Artificial Sequence
  Synthetic primer
tcactcgtctttttggccttc

SEQ ID NO: 11 - DNA, 32 bp, Artificial Sequence
  Synthetic primer
ctcatcgcggccgcgttcgcctcgagtgcttg

SEQ ID NO: 12 - DNA, 30 bp, Artificial Sequence
  Synthetic primer
ctcatcgaattcacgagccagtggaacgac

SEQ ID NO: 13 - DNA, 32 bp, Artificial Sequence
  Synthetic primer
ctcatcgttaacagcatctttatcgatgcgct

SEQ ID NO: 14 - DNA, 34 bp, Artificial Sequence
  Synthetic primer
ctcatcgttaactcactgacttcgttcgattttc

SEQ ID NO: 15 - DNA, 20 bp, Artificial Sequence
  Synthetic primer
cgattccttgagagcaactg

SEQ ID NO: 16 - DNA, 20 bp, Artificial Sequence
  Synthetic primer
gacgcagatgtgcgtgtatc

SEQ ID NO: 17 - DNA, 21 bp, Artificial Sequence
  Synthetic primer
actgccgtgtggtaaaatgaa

SEQ ID NO: 18 - DNA, 23 bp, Artificial Sequence
  Synthetic primer
gccatagagttcattgcggactc

SEQ ID NO: 19 - DNA, 21 bp, Artificial Sequence
  Synthetic primer
tcccctctgctggcgaaaagt

SEQ ID NO: 20 - DNA, 25 bp, Artificial Sequence
  Synthetic primer
agcgttcgtggtcaactatcgattg

SEQ ID NO: 21 - DNA, 24 bp, Artificial Sequence
  Synthetic primer
tcttcgttagctggcaactcacct

SEQ ID NO: 22 - DNA, 24 bp, Artificial Sequence
  Synthetic primer
atgacccagcagctaagcagatca

SEQ ID NO: 23 - protein, 46 aa, Homo sapiens
RSYGLCWTISITWELTELFFMHLLPNFAECWWDQVILDILLCNGGG

SEQ ID NO: 24 - protein, 46 aa, Cricetulus griseus; identical to SEQ ID NO: 23

SEQ ID NO: 25 - protein, 46 aa, Mus musculus; identical to SEQ ID NO: 23

SEQ ID NO: 26 - protein, 46 aa, Homo sapiens
RDWWMCMIISVMFEFLEYSLEHQLPNFSECWWDHWIMDVLVCNGLG

SEQ ID NO: 27 - protein, 46 aa, Cricetulus griseus
RDWWMCMIISVMFEFLEYSLEHQLPNFSECWWDHWIMDVLICNGLG

SEQ ID NO: 28 - protein, 46 aa, Mus musculus; identical to SEQ ID NO: 26

SEQ ID NO: 29 - protein, 46 aa, Trypanosoma cruzi
RDWRFATCVSITFEIIEITLQHALPNFKECWWDHLLLDVLLCNGGG

SEQ ID NO: 30 - protein, 46 aa, Leishmania amazonensis
RDWRMVTAVSLGFEVVEVTFQHVLPNFSECWWDHIVLDVVICNAGG

SEQ ID NO: 31 - protein, 46 aa, Cryptosporidium parvum
RNNFLVWFNSILFEWLEITLRHILPNFYECWWDHIILDIFGCNMIG

SEQ ID NO: 32 - protein, 46 aa, Plasmodium falciparum
RNFFLLNINSVIFELIELRFQHILPNFYECWWDHIFLDVLSCNLIG

SEQ ID NO: 33 - protein, 46 aa, Arabidopsis thaliana
RNQPLLWVLSIGFELLEVTFRHMLPNFNECWWDSIVLDILICNWFG

SEQ ID NO: 34 - protein, 46 aa, Neospora caninum
RDARLLWILSLLFEWMEISLRHILPNFWECWWDHLILDVFGCNLLG

SEQ ID NO: 35 - protein, 46 aa, Toxoplasma gondii
RDARLLWILSVLFEWMEISLRHILPNFWECWWDHLILDVFGCNLLG

SEQ ID NO: 36 - protein, 46 aa, Phytophthora infestans
RDWRLCWVLSIAFEILELALQFVIPDFQECWWDSLLLDLLGANMLG

SEQ ID NO: 37 - protein, 46 aa, Perkinsus marinus
RDWHMCWTYSFAFEFAELGLVWLVPEFQECWWDSLLMDVFGANFIG

SEQ ID NO: 38 - protein, 46 aa, Eimeria tenella
RSWPCALALSLLFEAAEASLHWLLPEFQECWWDSVVLDAVLSNLLG

SEQ ID NO: 39 - protein, 46 aa, Neospora caninum
RNWGLCLLYSIFFELGELSFHWLVPELCECWWDSIFIDALLSNVSG

SEQ ID NO: 40 - protein, 46 aa, Toxoplasma gondii
RHWGFCLIYSLCFELGELSFHWLVPELCECWWDSIFIDALLSNVCG

(19) United States
(12) Patent Application Publication
(10) Pub. No.: US 2017/0252418 A1
(43) Pub. Date: Sep. 7, 2017

(54) GENETICALLY MODIFIED COCCIDIAN PARASITES USEFUL AS VACCINES

(71) Applicants: HUMBOLDT-UNIVERSITÄT ZU BERLIN, Berlin (DE); UNIVERSITEIT UTRECHT HOLDING B.V., Utrecht (NL)

(72) Inventors: Nishith GUPTA, Berlin (DE); Ruben ARROYO-OLARTE, Berlin (DE); Richard LUCIUS, Berlin (DE); Jos BROUWERS, Utrecht (NL); J. Bernd HELMS, Utrecht (NL); Ildiko DUNAY, Berlin (DE)

(73) Assignees: HUMBOLDT-UNIVERSITÄT ZU BERLIN, Berlin (DE); UNIVERSITEIT UTRECHT HOLDING B.V., Utrecht (NL)

(21) Appl. No.: 15/510,933

(22) PCT Filed: Sep. 16, 2015
(86) PCT No.: PCT/EP2015/071168

§ 371 Date: Mar. 13, 2017

(30) Foreign Application Priority Data
Sep. 16, 2014 (EP) 14185015.6

(51) Int. Cl. A61K 39/012; C12N 1/36; C12N 1/10; C07F 9/10; C12N 9/12

(52) U.S. Cl. CPC A61K 39/012; C07F 9/106; C12N 9/1288; C12N 1/10; C12N 1/36; A61K 2039/522

(57) ABSTRACT

A genetically modified coccidian parasites wherein expression of phosphatidylthreonine synthase (PTS) is disrupted, a polynucleotide including a nucleotide sequence encoding a phosphatidylthreonine synthase (PTS) enzyme, which catalyzes the production of a lipid, phosphatidylthreonine (PtdThr). PtdThr is an exclusive, major and physiologically important lipid in selected coccidian parasites, which is required for a normal growth and virulence of coccidian parasites. Coccidian parasites, having the expression of PTS disrupted as described herein, are useful as vaccines. The phosphatidylthreonine synthase enzyme and the nucleotide encoding sequences thereof as well as the phosphatidylthreonine phospholipid can find use in diagnostic methods and diagnostic kits or in vaccine and drug development applications.

The present invention relates to coccidian parasites wherein expression of phosphatidylthreonine synthase (PTS) is disrupted, to a polynucleotide comprising a nucleotide sequence encoding a phosphatidylthreonine synthase (PTS), to a phosphatidylthreonine synthase (PTS) enzyme, and to a phosphatidylthreonine (PtdThr) lipid.

Coccidian parasites, having the expression of PTS disrupted as described herein, are useful as vaccines. The phosphatidylthreonine synthase enzyme and the nucleotide encoding sequences thereof as well as the phosphatidylthreonine phospholipid can find use in diagnostic methods and diagnostic kits or in vaccine and drug development applications.

Coccidian parasites, such as Toxoplasma, Eimeria, and Neospora species, are unicellular pathogens, which pose a major socioeconomic burden worldwide. Toxoplasma (e.g. Toxoplasma gondii) is a widespread parasite infecting humans as well as nearly all other warm-blooded organisms. The parasite is a substantial threat to domestic animals of food and economic value (e.g. sheep, and pig) as well as to human health. The parasite causes cerebral and ocular toxoplasmosis in immunosuppressed individuals and in neonates. It can also inflict congenital disease in humans and animals leading to spontaneous abortions and fetal abnormalities. Similarly, Eimeria (e.g. Eimeria tenella) and Neospora (e.g. Neospora caninum) are major concerns for the poultry industry (causing e.g., diarrhea in chickens) and for the cattle industry (causing e.g., abortions in cows), respectively. Similar to Toxoplasma, Neospora parasites also form tissue cysts in the nervous system, which cannot be cured by drugs available, and thus persist for the entire life of the infected host. These cysts can reactivate to cause potentially fatal acute infection upon decline in immune response such as by HIV-AIDS infection or during organ transplantations or just by aging in elderly population.

Thus, there is a clear need for preventive methods to tackle coccidian parasite infections, in particular the yet-incurable cyst (chronic) infection.

A commercial live vaccine against Toxoplasma is available (Toxovax), which comprises a live-attenuated S48 strain of Toxoplasma gondii (Buxton, D. (1993), Toxoplasmosis: the first commercial vaccine, Parasitology today (Personal ed.), 9(9), 335-7 (review); Verma R. and Khanna P. (2013) Development of Toxoplasma gondii vaccine: A global challenge, Hum Vaccin Immunother., 2013 Feb.; 9(2):291-3 (review)). This vaccine is used in veterinary applications. However, it has several disadvantages: it is expensive, causes side effects, has a short shelf-life, and is genetically uncharacterized. Also, it is not suitable for human use because it is susceptible to reverting to its pathogenic virulence.

The inventors have identified a novel enzyme (phosphatidylthreonine synthase, PTS) expressed in coccidian parasites which is absent in all mammalian hosts, and which produces an exclusive membrane lipid known as phosphatidylthreonine (PtdThr). It is noteworthy that PtdThr has been previously reported as a rare and notably minor PtdSer analog in certain mammalian cells and in selected prokaryotes (J. Mitoma, T. Kasama, S. Furuya, Y. Hirabayashi, Occurrence of an unusual phospholipid, phosphatidyl-L-threonine, in cultured hippocampal neurons. Exogenous L-serine is required for the synthesis of neuronal phosphatidyl-L-serine and sphingolipids., The Journal of Biological Chemistry 273, 19363-19366 (1998); P. T. Ivanova, S. B. Milne, H. A. Brown, Identification of atypical ether-linked glycerophospholipid species in macrophages by mass spectrometry., Journal Of Lipid Research 51, 1581-1590 (2010); F. D. Müller, S. Beck, E. Strauch, M. W. Linscheid, Bacterial predators possess unique membrane lipid structures., Lipids 46, 1129-40 (2011); L. Heikinheimo, P. Somerharju, Translocation of phosphatidylthreonine and -serine to mitochondria diminishes exponentially with increasing molecular hydrophobicity., Traffic 3, 367-77 (2002)), occasionally with rather detrimental effect on cell physiology (J. Mitoma, T. Kasama, S. Furuya, Y. Hirabayashi, Occurrence of an unusual phospholipid, phosphatidyl-L-threonine, in cultured hippocampal neurons. Exogenous L-serine is required for the synthesis of neuronal phosphatidyl-L-serine and sphingolipids., The Journal of Biological Chemistry 273, 19363-19366 (1998)).

It was shown that the base-exchange type phosphatidylserine synthase (PSS) in mammalian cells, normally using serine as its primary substrate, could also produce PtdThr as a byproduct under serine-deprived conditions. Under normal conditions, eukaryotic cells do not produce PtdThr in any detectable amounts. In contrast, the inventors reveal a surprisingly abundant and natural occurrence of PtdThr in a widespread eukaryotic pathogen, such as T. gondii and E. tenella.

The inventors have also found that the disruption of PTS abrogates the de novo synthesis of PtdThr, and impairs the asexual life cycle of coccidian parasites (e.g. T. gondii). In particular, the inventors have found that so modified parasites are avirulent and confer protection against parasite-inflicted diseases, such as acute and chronic toxoplasmosis in mice. The absence of PTS and of PtdThr being produced in mammalian cells means that the PTS-disrupted parasites cannot obtain sufficient PtdThr from the host cells to compensate for their PtdThr deficiency. This makes coccidian parasites with disrupted PTS expression rather avirulent and useful as vaccines, particularly for the farm and food animals, often infected by T. gondii.

Accordingly, the present invention relates to a coccidian parasite selected from Toxoplasma, Neospora and Eimeria species wherein the expression of phosphatidylthreonine synthase (PTS) enzyme is disrupted thereby reducing or eliminating the synthesis of phosphatidylthreonine (PtdThr). Suitable coccidian parasites include Toxoplasma, Neospora and Eimeria species. Toxoplasma gondii (also referred to as T. gondii or Tg), Neospora canium (also referred to as N. canium or Nc) and Eimeria tenella (also referred to as E. tenella or Et) may be particularly mentioned. Toxoplasma gondii may be of particular interest.

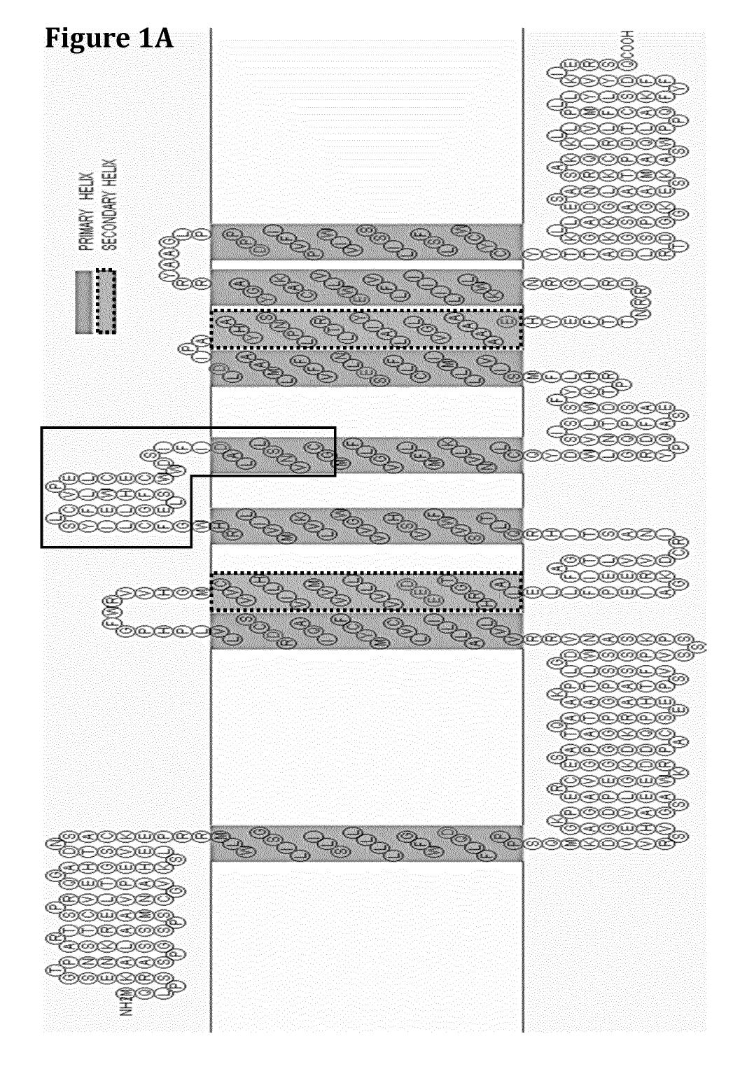

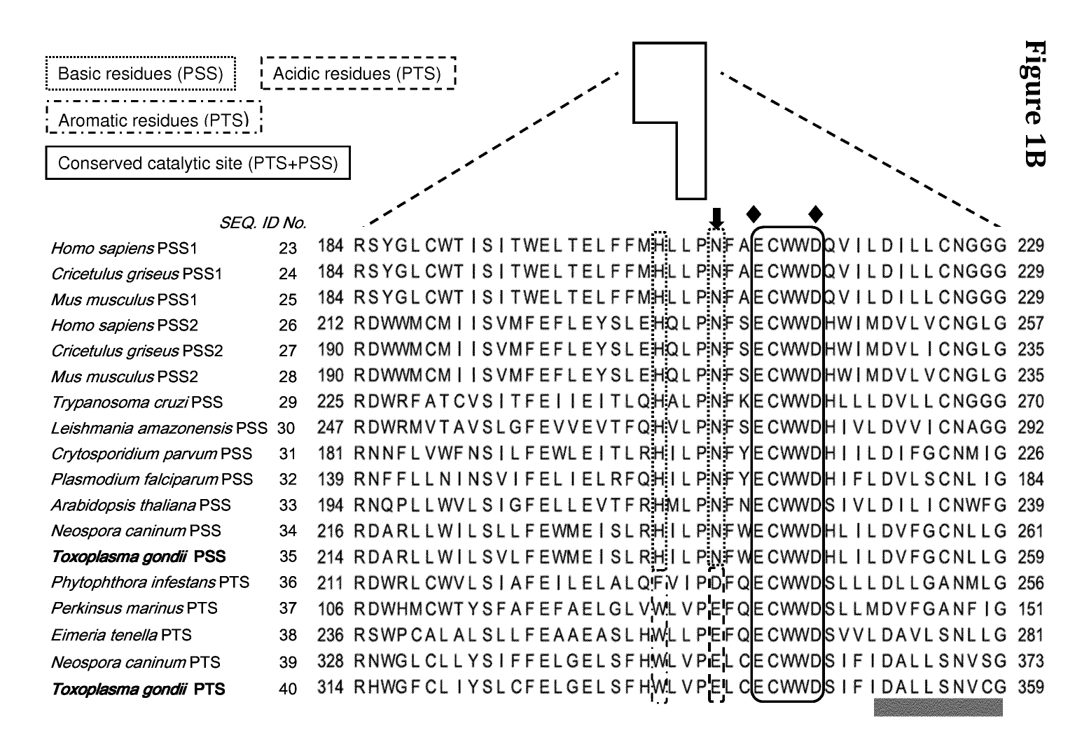

PTS is an enzyme expressed in coccidian parasites, which produces phosphatidylthreonine (PtdThr). This enzyme is distinct from the closely related and more ubiquitous phosphatidylserine synthase (PSS), which is also present in coccidian parasites. PSS produces phosphatidylserine (PtdSer) instead of phosphatidylthreonine (PdtThr). PTS and PSS share the same catalytic site, of sequence ECWWD, but differ in many other residues in the catalytic domain (FIG. 1). Of note is the fact that distinct asparagine, histidine and cysteine residues are strictly conserved in all PSS orthologs including in TgPSS, however not in TgPTS, which contains substitutions to glutamate, tryptophan and serine at the equivalent positions (FIG. 1).

The gene sequence encoding PTS, also referred to as PTS gene, is provided in SEQ ID No. 1 for Toxoplasma gondii, in SEQ ID No. 3 for Neospora caninum and in SEQ ID No. 5 for Eimeria tenella. The corresponding encoded amino acid sequence is provided in SEQ ID No. 2 for Toxoplasma gondii, in SEQ ID No. 4 for Neospora caninum and in SEQ ID No. 6 for Eimeria tenella.

PtdThr is a glycerophospholipid characterized by having a threonine bound to the phosphatidyl group via the OH group of threonine. Generally, PTS may produce a mixture of phosphatidylthreonine lipid species, whose structures may vary in the nature of the acyl groups attached to glycerol.

The structure of a phosphatidylthreonine (PtdThr) as described herein may be depicted by formula (I)

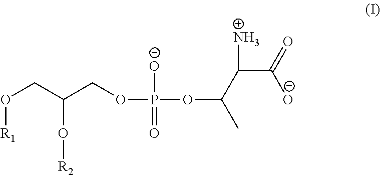

wherein R₁ and R₂ are independently selected from saturated and/or unsaturated acyl groups having from 8 to 46 carbon atoms, in particular from 10 to 36, more in particular from 14 to 26, and yet more in particular from 16 to 24 carbon atoms. Unsaturated acyl groups may have at least one carbon-to-carbon double bond, particularly 1 to 8, more in particular 1 to 6, and even more in particular 1 to 4 carbon-to-carbon double bonds. In several embodiments, R₁ and R₂ may be independently selected from unsaturated acyl groups having from 16 to 24 carbon atoms having 1 to 4 carbon-to-carbon double bonds. In particular embodiments, R₁ and R₂ may be independently selected from C20:1 and C20:4 acyl groups, more in particular, R₁ and may be an acyl group of formula CH₃—(CH₂)₇—(CH═CH)—(CH₂)₉—CO— and R₂ may be an acyl group of formula CH₃—(CH₂)₄—(CH═CH—CH₂)₄—(CH₂)₂—CO—.

In coccidian parasites as described herein, the expression of phosphatidylthreonine synthase (PTS) is disrupted. As indicated above, the disruption of PTS expression has been found to reduce or eliminate the synthesis of phosphatidylthreonine (PtdThr). The expression of PTS may be disrupted, e.g. by inactivating or deleting the gene encoding for the PTS enzyme. For instance, a coccidian parasite as described herein may be genetically modified.

Accordingly, in several aspects, the instant invention is directed to a method for preparing a genetically modified coccidian parasite, which comprises disrupting the expression of endogenous phosphatidylthreonine synthase (PTS) enzyme in a coccidian parasite selected from Toxoplasma, Neospora and Eimeria species by inactivating or deleting the corresponding PTS-encoding gene.

In the context of the invention, inactivation or deletion of a gene may be modification of a gene encoding a desired polypeptide (i.e. PTS) and/or a gene encoding a polypeptide involved in production of a primary or secondary metabolite by the parasite. In principle, reducing or eliminating the activity of the encoded protein can be done by targeted inactivation of the gene encoding PTS. The gene can be removed in its entirety. However, as an alternative also the deletion of part of the gene might result in a reduction or elimination of the activity of the encoded protein. For instance, the nucleotides encoding for the catalytic site of PTS may be removed or replaced, e.g. by a distinct nucleotide sequence, which lacks the nucleotides encoding for the catalytic site of PTS. The resulting genetically modified parasite will have the expression of PTS ablated. Consequently, the production of PtdThr can be reduced or eliminated. The catalytic site of PTS is highly conserved and comprises the amino acid sequence ECWWD. In particular, this catalytic sequence is found on positions 342-346 of the amino acid sequence of PTS for T. gondii and on positions 264-268 for E. tenella and 356-360 for N. caninum. The regions flanking the nucleotide sequence encoding for the catalytic site of PTS can be identified by standard DNA sequencing.

Alternatively, or additionally, nucleotide sequences responsible for the regulation or expression of the PTS-expressing gene, such as promoters enhancers and translational initiator sites can be modified or removed. Another way to influence the activity of PTS might be the modification of transport signals, if needed, or the introduction of anti-sense RNA.

Chromosomal modification is preferred since chromosomal modification will ensure a stable distribution of the functionality of the gene over the progeny cells. Deletion or disruption of a desired functionality in the chromosome (e.g. deletion or disruption of the sequence encoding PTS or encoding the catalytic site of PTS) can be done with non-homologous as well as with homologous recombination. Homologous recombination is preferred, as it opens the opportunity to introduce, to remove, or to simultaneously introduce and remove a functionality of choice. In particular, the gene expressing PTS may be disrupted by double homologous recombination.

When homologous recombination is intended, the transfected DNA constructs contain a DNA sequence that is homologous to a genomic target sequence of the specific parasite to be engineered. The skilled person will understand that 100% identity is not required to obtain homologous recombination. A percentage identity of 80%, preferably 90%, more preferably 95%, 98% or 99% will also suffice. Generally, the DNA sequence of interest to be inserted in the chromosome by homologous recombination is flanked by homologous sequences of a sufficient length to enable homologous recombination. Such a length may be at least about 300 bp in T. gondii, for instance between 300 and 3000 bp, depending on the parasite strains in use.

For the purpose of the present invention, the degree of identity between two amino acid sequences refers to the percentage of amino acids that are identical between the two sequences. The degree of identity is determined using the BLAST algorithm, which is described in Altschul, et al., (Journal of Molecular Biology, vol. 215, pp. 403-410 1990). Software for performing BLAST analyses is publicly available through the National Center for Biotechnology Information (http://www.ncbi.nlm.nih.gov/). The default settings for Blastp algorithm parameters are Expect threshold of 10, Word size of 3, Max matches in a query range of 0, Matrix is BLOSUM62, Gap Costs Existence of 11 and Extension of 1, Compositional adjustments at Conditional compositional score matrix adjustment.

For the purpose of the present invention, the degree of identity between two nucleotide sequences refers to the percentage of nucleotides that are identical between the two sequences. The degree of identity is determined using the BLAST algorithm, which is described in Altschul, et al., (Journal of Molecular Biology, vol. 215, pp. 403-410 1990). Software for performing BLAST analyses is publicly available through the National Center for Biotechnology Information (http://www.ncbi.nlm.nih.gov/). The default settings for Blastn algorithm parameters are Expect threshold of 10, Word size of 28, Max matches in a query range of 0, Match/Mismatch Scores of 1, −2, Gap Costs at Linear.

The sequences identifying the PTS genes in T. gondii, N. caninum and E. tenella, do not need to be 100% identical in order to modify the gene of interest by genetic engineering. In particular, the expression of PTS may be disrupted by inactivating or deleting a nucleotide sequence encoding an amino acid sequence of SEQ ID No. 2 (TgPTS), SEQ ID No. 4 (NcPTS) or SEQ ID No. 6 (EtPTS), or an homologous amino acid sequence (e.g. from other Eimeria or Neospora species) that displays a degree of identity of at least 30%, in particular at least 50%, more in particular at least 75%, even more in particular at least 90%, yet more in particular at least 95% and more in particular at least 99%.

Furthermore, the sequences in related parasites from other species or in parasites of particular strains might deviate from these sequences. However, making use of the PTS-expressing genes, sequences homologous to these genes, which have the same functionality, can be identified by those skilled in the art. Corresponding plasmid constructs can be prepared for performing homologous recombination in these strains. Thus, even if deviations from the sequences of the above-identified genes exist in a certain strain, homologous proteins can be identified using standard computational and wet-lab methods. For instance, the PTS activity of a given protein can be determined as explained below.

The parasites according to the present invention can be prepared using technologies known in the art. In particular, introducing DNA into parasites by electroporation (e.g. using a transfection instrument) may achieve transformation of coccidian parasites. Electroporation involves a high-voltage discharge passed through, e.g. a suspension containing coccidian parasites (for example Toxoplasma, Neospora or Eimeria species) and a suitable plasmid DNA comprising the desired functionality and/or homologous DNA sequences specific to the parasite in consideration.

The disruption of the PTS activity in a coccidian parasite as described herein may be tested by identifying the reduction or elimination of PtdThr production in the disrupted parasite when compared to the non-disrupted parasite (also referred to as wild-type or parental strain). In particular, lipid analyses may be used. For instance, the disrupted and the wild-type parasite strains may be grown in a suitable host (e.g. in human foreskin fibroblast (HFF) cells). The parasites can then be released from the host (e.g. using a syringe or by natural host-cell lysis by the parasite) and the lipids may be extracted from the parasites according to Bligh-Dyer (Bligh, E. G. & Dyer, W. J. “A rapid method of total lipid extraction and purification”, Canadian Journal Of Biochemistry And Physiology vol. 37, pp. 911-917, 1959). The extracted lipid fraction can be fractionated, e.g. by high-performance liquid chromatography (HPLC), and each lipid fraction can be analyzed and characterized, e.g. using HPLC coupled to a mass spectrometer (HPLC-MS), thereby identifying the presence or absence of PtdThr. Further, the amount of PtdThr and other lipids can be quantified by, e.g. performing two dimensional thin layer chromatography (2D-TLC) and a phosphorous analysis of the spots on the 2D-TLC as described by Rouser et al. (Two dimensional thin layer chromatographic separation of polar lipids and determination of phospholipids by phosphorus analysis of spots, Lipids, vol. 5, pp. 494-496, 1970).

Generally, the reduction in the total amount of PtdThr produced in a disrupted coccidian parasite as described herein when compared to the amount of PtdThr produced by the non-disrupted parasite (as measured by phosphorus quantification of 2D-TLC) may be up to 50 wt. %, in particular up to 25 wt. %, more in particular up to 15 wt. %, even more in particular up to 5 wt. %, yet more in particular up to 1 wt. % or even up to 0.1 wt. %. In several embodiments, disrupted coccidian parasites as described herein produce no PtdThr, or an amount of PtdThr, which is non-detectable by HPLC or 2D-TLC phosphorous analysis.

PTS-disrupted coccidian parasites as described herein are suitable for use as a vaccine. They may be used to vaccinate an animal or a human by administrating a coccidian parasite to said animal or human (e.g. by intra-peritoneal injection). Parasites as described herein may be preferably used to vaccinate an animal selected from sheep, pig, poultry and cattle. T. gondii may be a parasite of choice for sheep and pig vaccination in particular, E. tenella may be a parasite of choice for poultry vaccination in particular, and N. caninum may be a parasite of choice for cattle vaccination in particular. Of note is the fact that no chemotherapy and human vaccine are available to treat chronic toxoplasmosis. Therefore, prevention of zoonotic transmission by vaccinating the farm and food animals might be the best way forward to reduce and/or eradicate toxoplasmosis burden in human populations.

In several aspects the instant invention also relates to a polynucleotide comprising a nucleotide sequence encoding a phosphatidylthreonine synthase (PTS) enzyme.

A PTS-encoding polynucleotide may originate from, e.g. a coccidian parasite selected from Toxoplasma, Neospora and Eimeria species, or from a coccidian parasite selected from Toxoplasma gondii, Neospora caninum and Eimeria tenella. The coccidian parasite may preferably be Toxoplasma gondii.

Coccidian parasites, for instance, can be grown in a suitable medium to isolate the PTS-encoding polynucleotide, which can be cloned in a suitable vector for heterologous expression by methods known in the art. Suitable vectors include, for example, pQE60 for expression in Escherichia coli or pESC-Ura in Saccharomyces cerevisiae, which can be used for subsequent expression of PTS. As a mode of example pQE60 vector for overexpressing TgPTS in E. coli may be used.

A polynucleotide as described herein may also be produced synthetically by methods known in the art.

In several embodiments a polynucleotide as described herein may comprise a nucleotide sequence encoding a protein having an amino acid sequence of SEQ ID No. 2 for TgPTS, SEQ ID No. 4 for NcPTS or SEQ ID No. 6 for EtPTS, or homologues, which encode an amino acid sequence displaying at least 30% identity to the indicated sequences, in particular at least 50%, more in particular at least 75%, even more in particular at least 90%, yet more in particular at least 95% and more in particular at least 99% identity.

Such homologous sequences may encompass polymorphisms that may exist in cells from different populations or within a population due to natural or intra-strain variation. A homologue may further be derived from species other than the species where the specified DNA or amino acid sequence originates from, or may be artificially designed and synthesized. The PTS proteins designated as SEQ ID Nos. 2, 4 and 6 are encoded by the PTS polynucleotides of Toxoplasma gondii (SEQ ID No. 1), Neospora canium (SEQ ID No. 3) and Eimeria tenella (SEQ ID No. 5), respectively.

The PTS activity of a protein encoded by, e.g. T. gondii PTS gene or homologous sequences, can be demonstrated by overexpression of the gene in a suitable host (e.g. in a E. coli or yeast or mammalian cells) and subsequent identification of PtdThr by, e.g., isolation of the lipid fraction of the organism and its analysis to identify the presence of PtdThrby, e.g., thin layer chromatography (TLC) or high-performance liquid chromatography (HPLC) and mass spectrometry, or using other methods known in the art. For instance, overexpression of PTS can be performed by cloning in the pQE60 vector followed by transformation of E. coli.

A polynucleotide comprising a nucleotide sequence encoding a phosphatidylthreonine synthase (PTS) may be useful in the development of diagnostic methods and diagnostic kits or in the development of vaccines and drugs. For instance, a polynucleotide sequence as described herein may serve to express PTS, which in turn may be used for the production of PtdThr. For example, detection of PtdThr in samples from the infected patients or animals can be used to diagnose parasitic infections. Further, the availability of PTS and PtdThr can assist in the development of targeted drugs by, e.g. modification of the enzyme substrate threonine or of the product PtdThr with inhibitory properties and assessment PTS activity in the presence of the modified chemicals.

Accordingly, in several aspects the instant invention also relates to a phosphatidylthreonine synthase (PTS), encoded by the nucleotide sequences described above, and to a phosphatidylthreonine (PtdThr) lipid as also described above.

The instant invention is further illustrated by the following examples without being limited thereto or thereby.

### EXAMPLE 1—IDENTIFICATION AND CHARACTERIZATION OF PTDTHR

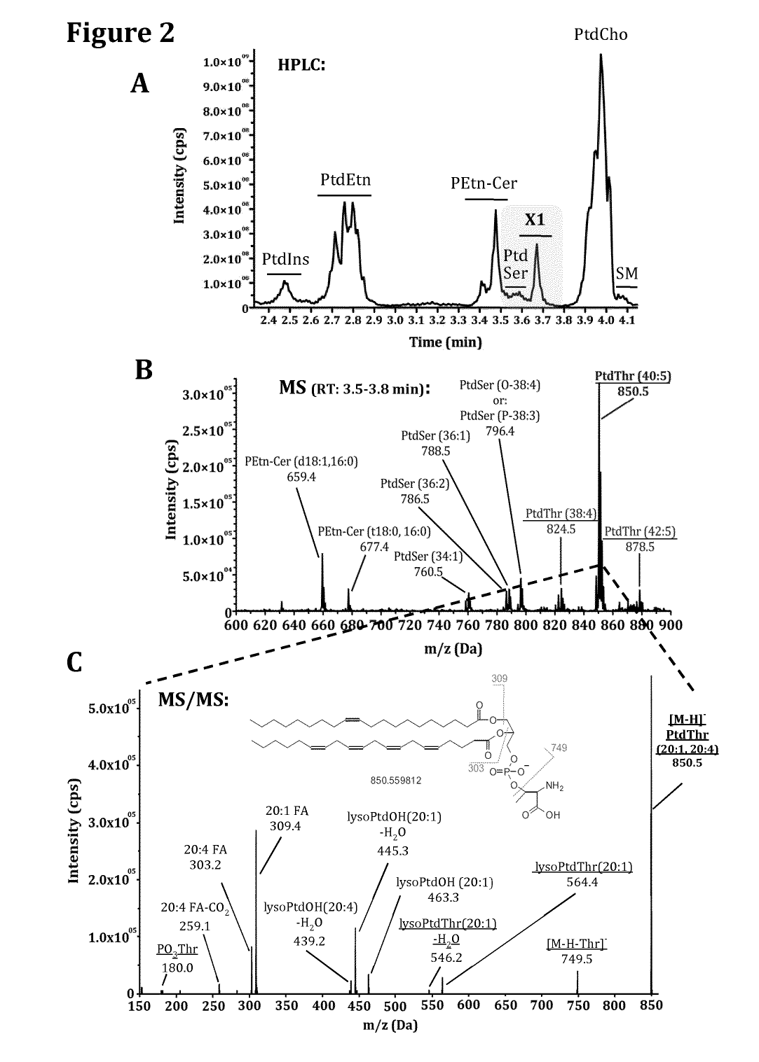

PtdThr was identified by HPLC fractionation of the lipids produced in T. gondii parasites as a major lipid peak. The acyl chain composition of PtdThr was determined by lipidomic analyses of the HPLC-derived fraction using mass spectrometry as described in detail below. The results are shown in FIG. 2. The structure of a PtdThr is shown in FIG. 2(C).

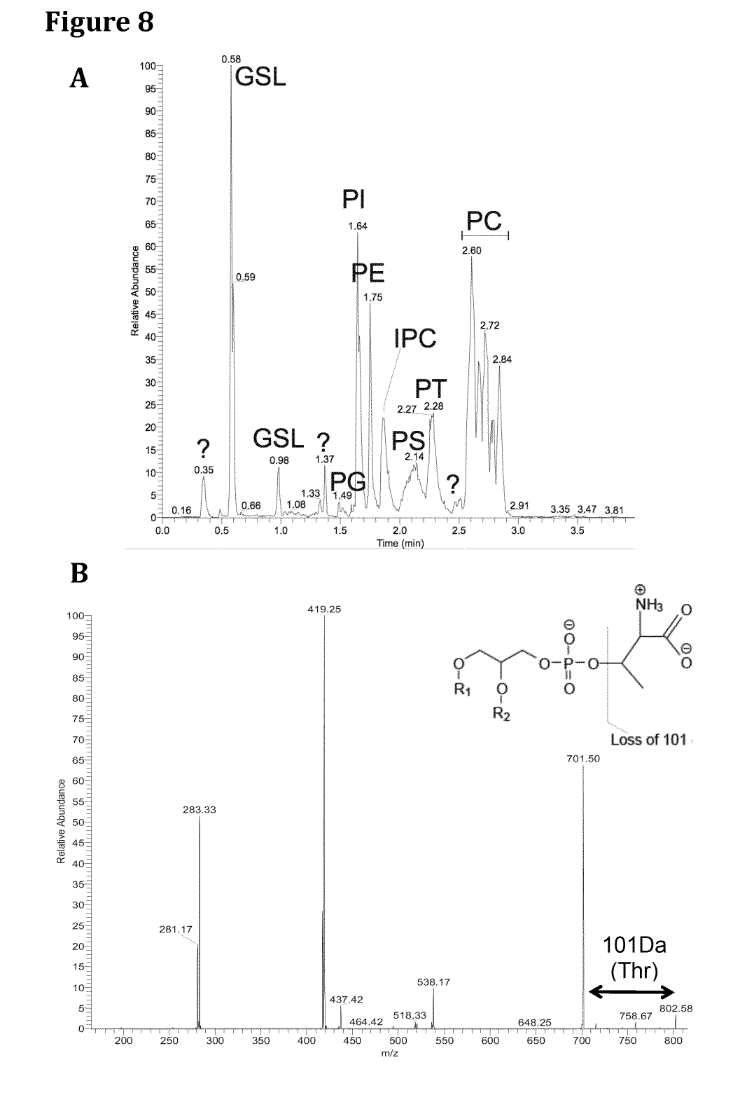

In a similar way PtdTHr was identified in E. tenella using chromatography and mass spectrometry (FIGS. 8 (A) and (B), wherein PtdThr is referred to as PT in FIG. 8(A)).

### Parasite and Host Cell Cultures

A Δku80 (type I) strain of T. gondii was provided by Vern B. Carruthers (University of Michigan, USA) (Huynh M H, Carruthers V B, agging of endogenous genes in a Toxoplasma gondii strain lacking Ku80, Eukaryot Cell. 2009 Apr.; 8(4):530-9). Tachyzoites of the Δku80 strain of T. gondii were propagated in human foreskin fibroblast (HFF) cells (obtained from ATCC, American type culture collection) using Dulbecco's Modified Eagle Medium (DMEM) containing fetal bovine serum (10%), glutamine (2 mM), minimum essential medium (MEM) non-essential amino acids (100 μm glycine, alanine, asparagine, aspartic acid, glutamic acid, proline, serine), sodium pyruvate (1 mM), penicillin (100 U/ml) and streptomycin (100 μg/ml) in a humidified incubator (37° C., 5% CO₂). Parasites were routinely cultured at a multiplicity of infection (MOI) of 3 every 2-3 days unless stated otherwise. HFF were harvested by trypsinization, and grown to confluence in fresh flasks, dishes or plates for infection assays as per experimental requirements.

### Lipid Extraction

Parasites were syringe-released from infected HFF (MOI, 3; 42-48 hours of infection) and passed twice through 23G and 27G needles. Host debris was removed by filtering the parasite suspension through a 5 μm filter (Merck Millipore, Germany). Cell pellets were re-suspended in 0.4 ml of PBS and lipids were extracted according to Bligh-Dyer (Bligh, E. G. & Dyer, W. J. “A rapid method of total lipid extraction and purification”, Canadian Journal Of Biochemistry And Physiology vol. 37, pp. 911-917, 1959). Briefly, 0.5 ml chloroform and 1 ml methanol were added to the samples, which were then vortexed, allowed to stand for 30 min and then centrifuged (2000 g, 5 min). The supernatant was transferred to a glass tube followed by addition of chloroform and 0.9% KCl (1 ml each). Samples were mixed, centrifuged and the lower chloroform phase containing lipids was transferred to a conical glass tube. Samples were stored at −20° C. in the airtight glass tubes flushed with nitrogen gas until further use.

### Lipidomics Analyses

Total lipids were fractionated on chloroform-equilibrated silica 60 columns. Neutral lipids were eluted by acetone washing of the column. Phospholipids were purified by 5× washing with 1 column-volume of chloroform/methanol/water (1:9:1). Each lipid fraction was collected, dried under nitrogen stream at 30° C., and stored at −20° C. for downstream assays. Lipidomics was performed using automated HPLC electrospray or atmospheric-pressure-ionization tandem mass spectrometry. Internal standard PtdCho (44:2) was mixed with extracted lipids to calibrate the recovery of major lipids. A 10-20 μl aliquot of phospholipid extract in chloroform and methanol (1:1) was introduced onto a HILIC column (Kinetex, 2.6 μm) at a flow rate of 1 ml/min to separate phospholipid classes. MS data were collected using either a 4000 QTRAP (AB Sciex, Concord) or a LTQ-XL (Thermo Scientific, Hampton) mass spectrometer. Data were processed using the proprietary software of the respective instrument manufacturers.

The main lipidomic results of T. gondii tachyzoites are shown in FIG. 2. FIG. 2 (A) shows the HPLC elution profile with the retention times and relative abundance of phospholipids isolated from extracellular parasites (10⁷). X1, eluting between 3.5-3.8 min, represents the lipid identified as PtdThr. FIG. 2 (B) shows the MS analysis of the X1 fraction revealing PtdThr, PtdSer and phosphoethanolamine-ceramide (PEtnCer) species. Individual lipids were identified by their fragmentation patterns and m/z ratios in the negative ionization mode. FIG. 2 (C) shows the MS/MS spectrum of the X1-derived peak (m/z 850) from FIG. 2(B). Note the neutral loss of 101 Da (850.5-749.5 transition) corresponding to the loss of polar head group threonine. The acyl chains were identified by their masses. The acyl chain of position sn-1 was identified as acyl 20:1 and the acyl of position sn-2 was identified as acyl 20:4. Sn-1 and sn-2 refer to the first and second carbon of the glycerol backbone in a given phospholipid. The third carbon contains the polar head group, such as threonine in PtdThr.

### EXAMPLE 2—IDENTIFICATION AND CHARACTERIZATION OF PTS

The genetic origin of PtdThr was determined by searching the parasite database Toxoplasma genomics resource database (ToxoDB) for expression of a relevant enzyme based on the structural similarity of threonine with serine. Two putative PtdSer synthases were found and their complete open reading frames were subsequently cloned (ToxoDB reference numbers TGGT1_273540 and TGGT1_261480). They were found to encode for 614 and 540 amino acid residues respectively. By sequence alignments with mammalian enzymes they were tentatively identified as close homologs of base-exchange type PtdSer synthases (PSS). The results of the alignment are presented in FIG. 1. Based on in silico analyses and the wet-lab results described herein, the two synthases were named TgPSS (Toxoplasma gondii phosphatidylserine synthase) and TgPTS (Toxoplasma gondii phosphatidylthreonine synthase). Unlike PSS occurring across the phyla, homologs of PTS could only be found in selected parasitic (Neospora, Eimeria, Phytophtora) and free-living (Perkinsus) chromalveolates organisms. Of note is the fact that distinct asparagine, histidine and cysteine residues are strictly conserved in all PSS orthologs including in TgPSS, however not in TgPTS, which contains substitutions to glutamate, tryptophan and serine at the equivalent positions (FIG. 1).

Molecular Cloning of T. gondii PTS

Parasites were syringe-released from infected HFF cells as described above for Example 1. The parasite RNA was isolated using Trizol-based method (Invitrogen) and was subsequently reverse-transcribed into first-strand cDNA.

The open reading frames of TgPSS and TgPTS were amplified from first-strand cDNA using PfuUltra II Fusion polymerase (Agilent Technologies). The primers used for amplification of the open reading frames are listed on table 1 (SEQ ID Nos. 7-10). The open reading frames were cloned into a commercial pDrive vector (Qiagen, Germany) following the manufacturer's protocol, which allowed DNA sequencing of the amplicons.

TABLE 1
PCR Primers for annotation of TgPTS and TgPSS open
reading frames

| SEQ ID | Primer | |
|---|---|---|
| Number | Name | Nucleotide Sequence |
| 7 | TgPTS-ORF-F | 5′-ATGCAACTCCCTTCAAGA-3′ |
| 8 | TgPTS-ORF-R | 5′-TCACTGACTTCGTTCCATTTTCACG-3′ |
| 9 | TgPSS-ORF-F | 5′-ATGTGTCGGGGACCGCCGCT-3′ |
| 10 | TgPSS-ORF-R | 5′-TCACTCGTCTTTTTGGCCTTC-3′ |

### EXAMPLE 3—GENETIC ABLATION OF PTS IN T. GONDII

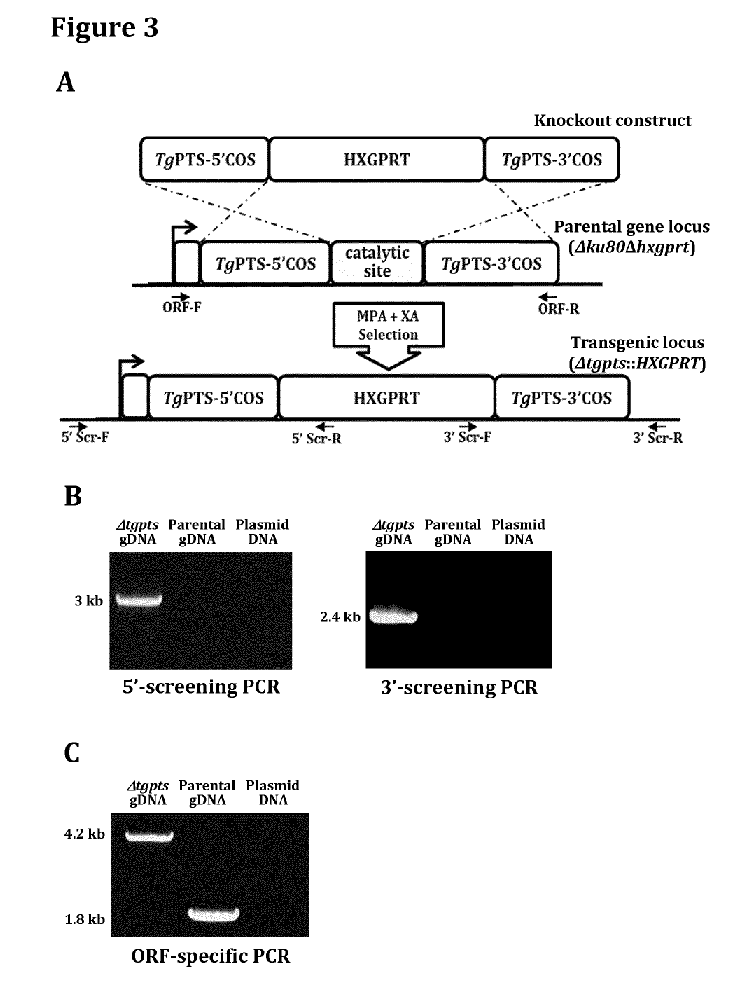

The PTS gene of T. gondii (TgPTS) was disrupted by double homologous cross over. A knockout plasmid was constructed, which contained 5′ and 3′ crossover sequence (COS) of TgPTS flanking a hypoxanthine xanthine guanine phosphoribosyltransferase (HXGPRT) marker as depicted in FIG. 3 (A). The HXGPRT marker was used to allow transgenic selection by mycophenolic acid (MPA) and xanthine (XA) (Donald, et al. “Insertional tagging, cloning, and expression of the Toxoplasma gondii hypoxanthine-xanthine-guanine phosphoribosyltransferase gene. Use as a selectable marker for stable transformation”, The Journal of Biological Chemistry, vol. 271, pp. 14010-14019, 1996).

### Knockout Vector Construction

The 5′ and 3′ crossover sequences (5′COS, 3′COS) of TgPTS were amplified using the genomic DNA isolated from fresh extracellular tachyzoites and using the primers of Table 2 (SEQ ID Nos. 11-14).

The 5′COS (0.9 kb) and 3′COS (0.8 kb) were cloned at NotI/EcoRI and HpaI/HpaI sites of a pTKO-HXGPRT vector, respectively. The resulting knockout vector contained 5′ and 3′COS of the TgPTS gene flanking a hypoxanthineguanine-phosphoribosyltransferase (HXGPRT) selection marker (pTKO-5′COS-HXGPRT-3′COS).

TABLE 2
PCR Primers for cloning of the TgPTS-5′COS and the
TgPTS-3′COS in the resulting TgPTS knockout vector

| | Primer Name | |
|---|---|---|
| SEQ ID | (restriction | Nucleotide Sequence |
| Number | site) | (restriction site underlined) |
| 11 | TgPTS-5′COS-F | 5′-CTCATCGCGGCCGCGTTCGCCTCGAGTGCTTG-3′ |
| | (NotI) | |
| 12 | TgPTS-5′COS-R | 5′-CTCATCGAATTCACGAGCCAGTGGAACGAC-3′ |
| | (EcoRI) | |
| 13 | TgPTS-3′COS-F | 5′-CTCATCGTTAACAGCATCTTTATCGATGCGCT-3′ |
| | (HpaI) | |
| 14 | TgPTS-3′COS-R | 5′-CTCATCGTTAACTCACTGACTTCGTTCGATTTTC-3′ |
| | (HpaI) | |

Genetic Modification of T. gondii

The knockout plasmid constructs (also referred to as knockout vector) were transfected into fresh tachyzoites (Δku80) suspended in Cytomix (120 mM KCl, 0.15 mM CaCl₂, 10 mM K₂HPO₄/KH₂PO₄ pH 7.6, 25 mM HEPES pH 7.6, 2 mM EGTA, 56 mM MgCl₂) using a BTX instrument (BTX Harvard Apparatus, USA) with the following conditions: 50 μg DNA, about 10⁷ parasites, 2 kV, 50Ω, 25 ρF and 250 μs. The knockout vector (pTKO-5′COS-HXGPRT-3′COS) permitted disruption of the TgPTS gene to generate a Δtgpts mutant by mycophenolic acid (25 μg/ml) and xanthine (50 μg/ml) selection.

Single (clonal) drug-resistant mutant parasites were isolated by the limiting dilution method. The isolated PTS gene-disrupted parasites (i.e. lacking the conserved catalytic (ECWWD) site, also referred to as the Δtgpts mutant or Δtgpts strain), were screened for 5′- and 3′-crossover events at the TgPTS gene locus by PCR, using screening primers 5′Scr-F/R and 3′Scr-F/R (Table 3, SEQ ID Nos. 15-18) pDrive vector (Qiagen, Germany) for standard cloning and DNA sequencing.

TABLE 3
PCR primers for 5′ and 3′ recombination screening
of the Δtgpts mutant

| SEQ ID | | |
|---|---|---|
| Number | Primer Name | Nucleotide Sequence |
| 15 | TgPTS-KO-5′Scr-F | 5′-CGATTCCTTGAGAGCAACTG-3′ |
| 16 | TgPTS-KO-5′Scr-R | 5′-GACGCAGATGTGCGTGTATC-3′ |
| 17 | TgPTS-KO-3′Scr-F | 5′-ACTGCCGTGTGGTAAAATGAA-3′ |
| 18 | TgPTS-KO-3′Scr-R | 5′-GCCATAGAGTTCATTGCGGACTC-3′ |

The results of the screening are presented in FIG. 3 (B), which displays PCR images of a representative clonal Δtgpts strain showing specific amplification of 3-kb and 2.4-kb long DNA bands by 5′ and 3′ screening. The parental gDNA and knockout plasmid were included as negative controls, respectively.

The Δtgpts mutant parasites were validated by the successful insertion of the selection marker (HXGPRT) at the TgPTS locus by ORF-specific PCR using the primers TgPTS-ORF-F and TgPTSORF-R of Table 1 (SEQ ID Nos. 7-10).

The results are presented in FIG. 3 (C), which displays the resulting ORF-specific PCR confirming a successful insertion of the selection marker in the PTS gene. The Δtgpts cDNA shows amplification of an expected 4.2-kb band as opposed to the 1.8-kb band in the parental cDNA, and none in the plasmid DNA, which corroborated the targeted insertion of selection marker and deletion of the predicted catalytic site (342ECWWD346).

The identity of all PCR amplicons was confirmed by sequencing, which was performed by the genomics company LGC Limited (Germany) using standard DNA sequencing methods.

### EXAMPLE 4—EFFECT OF PTS ABLATION ON PTDTHR PRODUCTION IN T. GONDII

The phospholipid composition of the Δtgpts and parental strains was investigated by thin layer chromatography (TLC), lipid phosphorus assays and lipidomics analyses. The total lipids were isolated from parasites as explained above for Example 1.

### Thin Layer Chromatography and Phosphorus Quantification

Lipids were resolved by two-dimensional TLC on silica 60 plates (Merck, Germany) using chloroform/methanol/ammonium hydroxide (65:35:5) and chloroform/acetic acid/methanol/water (75:25:5:2.2) as the solvents. They were visualized by staining with iodine vapors and/or ninhydrin spray, and were identified based on their co-migration with authentic standards (Avanti Lipids). The major iodine-stained phospholipid bands were scraped off the TLC plate, and quantified by phosphorus assay as described elsewhere (Rouser et al., “Two dimensional thin layer chromatographic separation of polar lipids and determination of phospholipids by phosphorus analysis of spots”, Lipids, vol. 5, pp. 494-496, 1970).

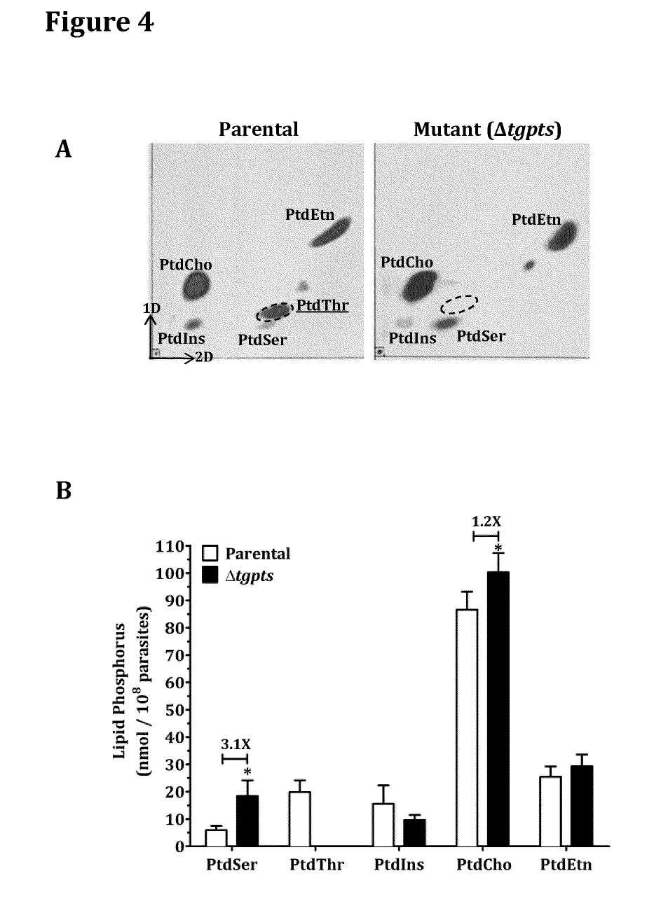

The results are presented in FIG. 4.

FIG. 4 (A) shows the lipid profiles of the indicated strains by two-dimensional TLC. The total lipids (0.8-1×10⁸ parasites) were resolved and detected by iodine vapor staining. It is noted that the PdtThr band is absent in the Δtgpts mutant and that an increased production of PtdSer is also observed. FIG. 4 (B) shows the lipid phosphorus measurements of bands from the TLC plates of FIG. 4 (A) showing the lack of PtdThr and induction of PtdSer in the Δtgpts mutant. The reproducibility of the results was confirmed in four independent biological experiments (n=4).

As expected, the parental parasites harbored a notable amount of PtdThr (FIGS. 4 A & B). Synthesis of PtdThr was completely abrogated in the Δtgpts strain. Concurrently, a three-fold increase in PtdSer was observed, whereas other lipids remained largely unaffected (FIG. 4B). Genetic complementation of the mutant with a functional TgPTS recovered PtdThr, as well as reversed PtdSer content to the normal level.

Lipidomic Analyses

The lipidomic analyses using HPLC/MS as described above for Example 1.

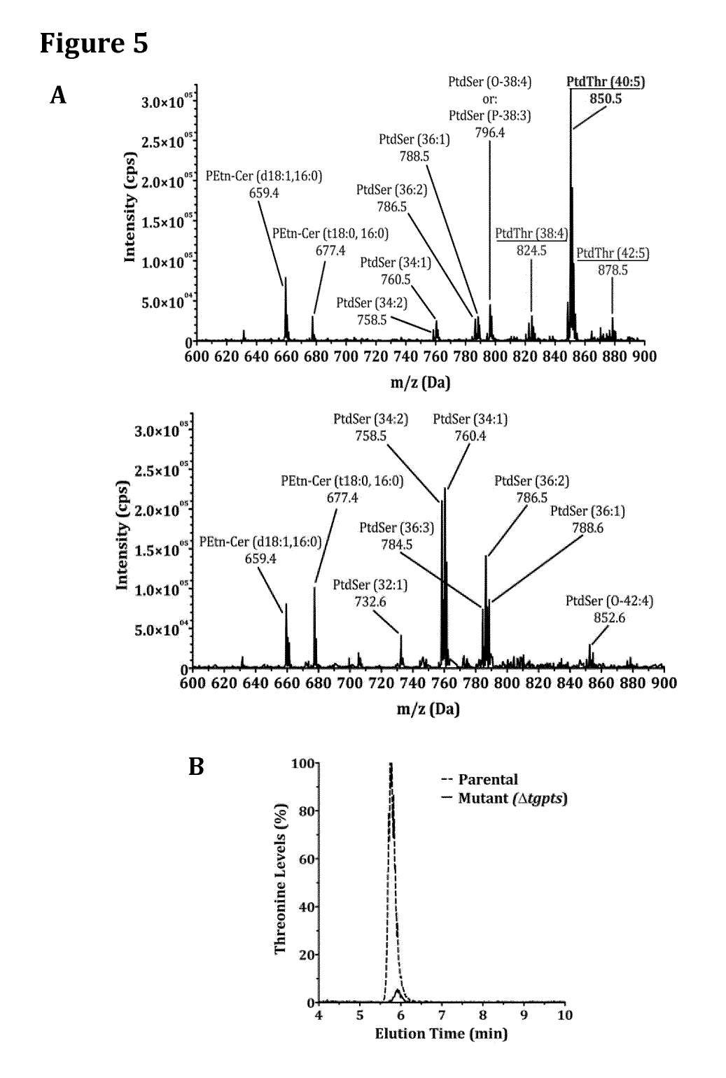

The results are presented in FIG. 5. The lipidomics results confirmed the absence of all PtdThr-derived species in the mutant. In particular, FIG. 5 (A) shows the representative MS profiles of the HPLC-resolved lipid (retention time, 3.5-3.8 min) from the parental and Δtgpts strains (˜107 parasites each) confirming the presence of a major (m/z 850.5, 40:5) and two minor (m/z 824.5, 38:4; m/z 878.5, 42:5) PtdThr species in the parental parasites, which are completely absent in the mutant. It is noted that PtdSer peaks are more intense in the Δtgpts sample, which is in agreement with lipid analysis by TLC and chemical phosphorus assays (FIG. 4).

Taken together, these results show an autonomous synthesis of PtdThr by TgPTS enzyme, and the loss of PtdThr lipid following its functional ablation in T. gondii.

### EXAMPLE 5—EFFECT OF PTS ABLATION IN T. GONDII ON GLIDING MOTILITY AND VIRULENCE OF T. GONDII

### Gliding Motility Assays

Parasites (syringe-released from infected HFF as explained above) were incubated on BSA (0.01%)-coated coverslips in Hanks Balanced Salt Solution (HBSS) for 15 min at 37° C. Samples were fixed in 4% paraformaldehyde and 0.05% glutaraldehyde (10 min), and stained with anti-TgSag1 primary antibody (donated by Jean-Franois Dubremetz, University of Montpellier, France) and Alexa488-conjugated secondary antibody (Life Technologies, Germany). The motile fraction and the trail length were quantified using the ImageJ software (National Institute of Health, USA).

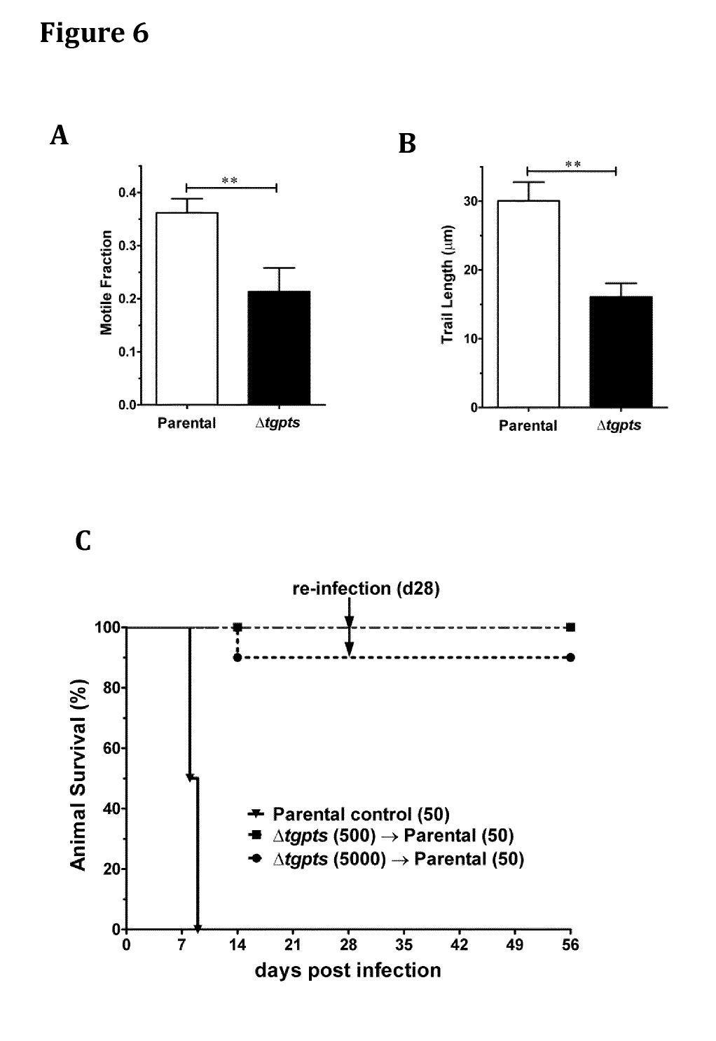

The results are shown in FIGS. 6 (A & B).

FIG. 6 (A) shows the motile fractions of different strains on BSA-coated glass coverslips. In total, 500-1000 parasites from 6 independent assays were scored for the presence or absence of a motility trail (immuno-stained with anti-TgSagl antibody) to calculate the motile fractions for each strain. FIG. 6 (B) shows the motility of individual parasite strains, as deduced by the length of TgSagl-stained trails. The error bars for FIGS. 6 (A & B) indicate the mean±SEM. *p<0.05, **p<0.01, and ***p<0.001.

As depicted in FIGS. 6 (A & B), the Δtgpts strain showed a distinguished reduction in the motile fraction and the trail length when compared to the parental. The strain complemented with PTS activity showed a reverted phenotype similar to the parental strain, ascertaining the specificity of the observation. Such reduced parasite motility eventually causes a poor invasion and egress of/from HFF host cells by the Δtgpts mutant. A collective defect in motility, invasion and egress impairs the lytic cycle and virulence of the Δtgpts mutant.

### In Vivo Parasite Infection for Virulence Testing

C57BL/6 mice (obtained from Janvier Labs, Saint Berthevin, France) were infected with tachyzoites of the Δku80 (parental) or Δtgpts strains. Parasites for in vivo infections were propagated in HFF cells; fresh host-free tachyzoites were released 40 hrs post-infection and filtered (5 μm), as described above. They were injected via intra-peritoneal route (50 parasites of the parental strain and 5×10² or 5×10³ parasites of the Δtgpts strain). 12 animals were monitored for the mortality and morbidity 3 times a day over a period of 4 weeks. An inoculum of 50 parental tachyzoites was used to challenge the Δtgpts-infected surviving animals, which were monitored for additional 4 weeks. A control group of naïve mice (n=4) was also infected with the same parental inoculum. The results are shown in FIG. 6 (C).

Quantification of T. gondii in the Mouse Brain

Cysts of the ME49 strain of T. gondii were harvested from the brains of female NMRI mice infected with T. gondii cysts 5 to 6 months earlier intraperitoneally as described elsewhere (Agrawal, G. G. van Dooren, W. L. Beatty, B. Striepen, Genetic evidence that an endosymbiont-derived endoplasmic reticulum-associated protein degradation (ERAD) system functions in import of apicoplast proteins, The Journal of Biological Chemistry 284, 33683-91 (2009)). The Δtgpts-vaccinated mice (500 parasites) were challenged with the ME49 T. gondii strain (3 cysts in 200 μl) parasites 4 weeks after vaccination. A control group of naïve animals was also included. Parasite burden in the mouse brain was estimated by counting cysts and semi-quantitative real-time PCR following another 4 weeks of infection with the ME49 T. gondii strain. Brain tissue was mechanically homogenized in 1 ml sterile phosphate-buffered saline and the cysts were counted using a light microscope. For quantitative PCR (qPCR), perfused brain tissue samples were snap-frozen and stored at −80° C. 30 mg tissue was used to purify nucleic acids (QIAgen, Germany). FastStart Essential DNA Green Master (Roche, Germany) was mixed with genomic DNA (90 ng) in triplicate reactions, which were developed in a LightCycler® 480 Instrument II (Roche, Germany). The parasite burden (target: TgB1 gene) was analyzed relative to mouse (reference: argininosuccinate lyase (MmASL)) by estimating target-to-reference ratio (LightCycler® 480 software v1.5.0). Primers used to amplify the TgB1 and MmASL genes are listed in Table 4 (SEQ ID Nos. 19-22).

TABLE 4
PCR primers for amplification of the TgB1 and MmASL
genes

| SEQ ID | | |
|---|---|---|
| Number | Primer Name | Nucleotide Sequence |
| 19 | TgB1-F | 5′-TCCCCTCTGCTGGCGAAAAGT-3′ |
| 20 | TgB1-R | 5′-AGCGTTCGTGGTCAACTATCGATTG-3′ |
| 21 | MmASL-F | 5′-TCTTCGTTAGCTGGCAACTCACCT-3′ |
| 22 | MmASL-R | 5′-ATGACCCAGCAGCTAAGCAGATCA-3′ |

All in vivo assays were in compliance with the German animal protection laws directed by Landesverwaltungsamt Sachsen-Anhalt, Germany.

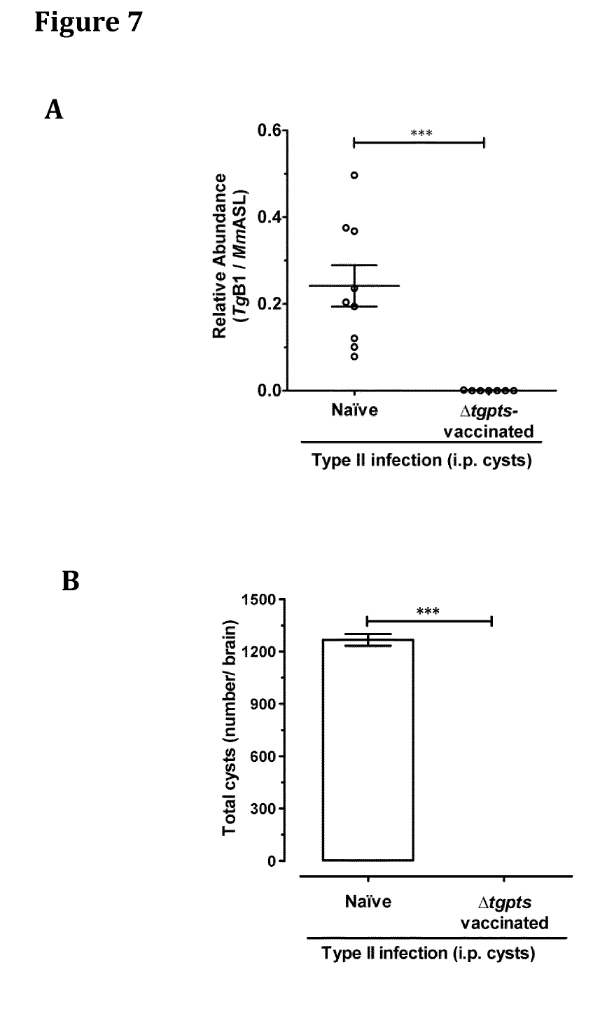

The results are shown in FIG. 7.

Examination of virulence in a murine model demonstrated that nearly all animals infected with the Δtgpts mutant survived as opposed to the parental strain that was explicitly lethal (FIG. 6C). Importantly, all mice enduring the mutant infection became categorically resistant to a subsequent lethal challenge by a hypervirulent Δku80 strain of T. gondii (type I infection) causing acute toxoplasmosis (FIG. 6C). To further expand the utility of our strain as a potential vaccine against chronic infection, the Δtgpts-infected animals were challenged with the cyst-forming ME49 T. gondii strain (type II infection). Surprisingly, in contrast to naïve animals, the mutant-vaccinated mice showed no signs of chronic stage cysts in their brain tissue (FIG. 7 A-B). Consistently, unlike the infected naïve control mice, no inflammatory lesions in the cortex or meninges of the Δtgpts-immunized animals were observed. In brief, these results demonstrate the in vivo requirement of PtdThr for the parasite virulence, and illustrate the prophylactic potential of a metabolically attenuated whole-cell ‘vaccine’ against acute as well as chronic toxoplasmosis.

1. A coccidian parasite selected from Toxoplasma, Neospora and Eimeria species wherein the expression of endogenous phosphatidylthreonine synthase (PTS) enzyme is disrupted thereby reducing or eliminating the synthesis of a phosphatidylthreonine (PtdThr).

2. The coccidian parasite of claim 1 which is selected from Toxoplasma gondii, Neospora caninum and Eimeria tenella, or is Toxoplasma gondii.

3. The coccidian parasite of claim 1 wherein the PtdThr is a PtdThr of formula (I):
(I)
wherein R1 and R2 are independently selected from satu-rated and/or unsaturated acyl groups having from 8 to 46 carbon atoms.

4. The coccidian parasite of claim 1, wherein the expression of PTS enzyme is disrupted by inactivating or deleting the corresponding PTS-encoding gene.

5. The coccidian parasite of claim 1 wherein the expression of PTS enzyme is disrupted by inactivating or deleting a nucleotide sequence encoding a protein sequence with at least 30% identity to Toxoplasma gondii PTS (SEQ ID No. 2), Neospora canium PTS (SEQ ID No. 4) or Eimeria tenella PTS (SEQ ID No. 6).

6. The coccidian parasite of claim 1, wherein the expression of PTS enzyme is disrupted by deleting or replacing the polynucleotide sequence, which comprises the nucleotides encoding for the catalytic site of PTS.

7. The coccidian parasite of claim 1 for use as a vaccine.

8. A method for preparing a genetically modified coccidian parasite of claim 1, which comprises disrupting the expression of endogenous phosphatidylthreonine synthase (PTS) enzyme in a coccidian parasite selected from Toxoplasma, Neospora and Eimeria species by inactivating or deleting the gene encoding PTS thereby reducing or eliminating the synthesis of phosphatidylthreonine (PtdThr).

9. The method of claim 8, which comprises inactivating or deleting the gene encoding PTS enzyme by single or double homologous recombination.

10. A polynucleotide comprising a nucleotide sequence encoding a phosphatidylthreonine synthase (PTS) enzyme.

11. The polynucleotide of claim 10 comprising a nucleotide sequence encoding a PTS enzyme having an amino acid sequence with at least 30% identity to Toxoplasma gondii PTS (SEQ ID No. 2), Neospora caninum PTS (SEQ ID No. 4) or Eimeria tenella PTS (SEQ ID No. 6).

12. A phosphatidylthreonine synthase (PTS) enzyme encoded by the nucleotide sequence of claim 10.

13. A phosphatidylthreonine (PtdThr) lipid of formula (I): 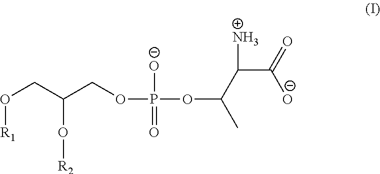
wherein R1 and R2 are independently selected from saturated and/or unsaturated acyl groups having from 8 to 46 carbon atoms.

14. The phosphatidylthreonine of claim 13 wherein R1 and R2 independently selected from saturated and/or unsaturated acyl groups having from 16 to 24 carbon atoms.

15. A method to vaccinate an animal or a human by administrating the coccidian parasite of claim 1 to said animal or human.

16. The method of claim 15 to vaccinate an animal selected from sheep, pig, poultry and cattle.
